(19) United States
(12) Patent Application Publication
(10) Pub. No.: US 2005/0222238 A1
(43) Pub. Date: Oct. 6, 2005

(54) Deuterated substituted pyrazolylbenzylsulfonamides and medicaments comprising said compounds

(76) Inventor: Rudolf-Giesbert Alken, Zepernick (DE)

Correspondence Address: KRIEGSMAN & KRIEGSMAN, 665 FRANKLIN STREET, FRAMINGHAM, MA, 01702, US

(21) Appl. No.: 10/498,708

(22) PCT Filed: Dec. 11, 2002
(86) PCT No.: PCT/DE02/04600

§ 371 Date: May 20, 2005

(30) Foreign Application Priority Data
Dec. 12, 2001 (DE) 101 62 121.3

(51) Int. Cl. A61K 31/415; C07D 231/12

(52) U.S. Cl. 514/406; 548/377.1

(57) ABSTRACT

The invention concerns deuterated substituted pyrazolyl benzylsulfonamides as well as pharmaceuticals containing these compounds. In addition, the invention concerns the use of deuterated substituted pyrazolyl benzylsulfonamides for the treatment of symptoms of osteoarthritis and rheumatoid arthritis as well as for the prevention and treatment of neoplasia, in particular adenomatous colorectal polyps in familial adenomatous polyposis, for the treatment of pain, in particular acute pain and dysmenorrhea, in particular primary dysmenorrhea. In addition, the invention discloses pharmaceutical compositions, which contain deuterated substituted pyrazolyl benzylsulfonamides as well as their physiologically compatible salts, in addition to pharmaceutically compatible adjuvants and/or additives, for the treatment of symptoms of osteoarthritis and rheumatoid arthritis as well as for the prevention and treatment of neoplasia, in particular adenomatous colorectal polyps in familial adenomatous polyposis, for the treatment of pain, in particular acute pain and dysmenorrhea, in particular primary dysmenorrhea.

The invention concerns deuterated substituted pyrazolyl benzylsulfonamides and pharmaceuticals containing these compounds.

A known representative of substituted pyrazolyl benzylsulfonamides is Celecoxib (EP 731,795, U.S. Pat. No. 5,466,823, U.S. Pat. No. 5,563,165, U.S. Pat. No. 5,760,068, U.S. Pat. No. 5,972,986), which is utilized for the treatment of symptoms of osteoarthritis and rheumatoid arthritis as well as for the prevention and treatment of neoplasia, in particular adenomatous colorectal polyps in familial adenomatous polyposis.

The object of the present invention is to prepare substituted pyrazolyl benzylsulfonamides which have improved pharmacokinetic and/or pharmacodynamic properties when compared with compounds already known.

It has now been found surprisingly that the deuterated substituted pyrazolyl benzylsulfonamides according to the invention have essentially better pharmacokinetic and/or pharmacodynamic properties than the undeuterated compounds.

According to the invention the object is thus solved by the preparation of deuterated substituted pyrazolyl benzylsulfonamides of the general formula I: 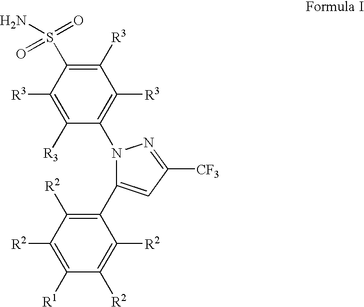
wherein R¹ is methyl or partially or completely deuterated methyl, R², independent of one another, indicates H or D, R³, independent of one another, is H or D, and wherein at least one of the groups R¹ to R³ is D or contains D.

Deuterated substituted pyrazolyl benzylsulfonamides according to the general formula I are preferred, wherein R¹ is partially or completely deuterated methyl, R², independent of one another, indicates H or D, and R³, independent of one another, is H or D.

Deuterated substituted pyrazolyl benzylsulfonamides according to the general formula I are particularly preferred, wherein R¹ is methyl or partially or completely deuterated methyl, R² indicates deuterium and R³, independent of one another, is H or D.

In particular, deuterated substituted pyrazolyl benzylsulfonamides according to the general formula I are preferred, wherein R¹ is methyl or partially or completely deuterated methyl, R², independent of one another, indicates H or D, and R³ is deuterium.

Deuterated substituted pyrazolyl benzylsulfonamides according to the general formula I are advantageous, wherein R¹ is partially or completely deuterated methyl, R² indicates deuterium and R³, independent of one another, is H or D.

Deuterated substituted pyrazolyl benzylsulfonamides of the general formula I are particularly advantageous, wherein R¹ is methyl or partially or completely deuterated methyl and R² and R³ indicate deuterium.

In particular, deuterated substituted pyrazolyl benzylsulfonamides according to the general formula I are particularly advantageous, wherein R¹ is partially or completely deuterated methyl, R², independent of one another, indicates H or D, and R³ is deuterium.

In addition, deuterated substituted pyrazolyl benzylsulfonamides according to the general formula I are advantageous, wherein R¹ is partially or completely deuterated methyl and R² and R³ indicate deuterium.

The following deuterated substituted pyrazolyl benzylsulfonamides are particularly advantageous according to the invention:
4-[5-(4-trideuteromethylphenyl)-3-trifluoromethylpyrazol-1-yl]benzylsulfonamide,
4-[5-(2,3,5,6-tetradeutero-4-methylphenyl)-3-trifluoromethylpyrazol-1-yl]benzylsulfonamide,
2,3,5,6-tetradeutero-4-[5-(4-tolyl)-3-trifluoromethylpyrazol-1-yl]benzylsulfonamide,
4-[5-(2,3,5,6-tetradeutero-4-trideuteromethylphenyl)-3-trifluoromethylpyrazol-1-yl]benzylsulfonamide,
2,3,5,6-tetradeutero-4-[5-(2,3,5,6-tetradeutero-4-methylphenyl)-3-trifluoromethylpyrazol-1-yl]benzylsulfonamide,
2,3,5,6-tetradeutero-4-[5-(4-trideuteromethylphenyl)-3-trifluoromethylpyrazol-1-yl]benzylsulfonamide,
2,3,5,6-tetradeutero-4-[5-(2,3,5,6-tetradeutero-4-trideuteromethylphenyl)-3-trifluoro methyl-pyrazol-1-yl]benzylsulfonamide.

The use of the deuterated substituted pyrazolyl benzylsulfonamides according to the invention as well as their physiologically compatible salts is preferred for the treatment of symptoms of osteoarthritis and rheumatoid arthritis as well as for the prevention and treatment of neoplasia, in particular adenomatous colorectal polyps in familial adenomatous polyposis, for the treatment of pain, in particular acute pain and dysmenorrhea, in particular primary dysmenorrhea.

In addition, the use of the deuterated substituted pyrazolyl benzylsulfonamides according to the invention as well as their physiologically compatible salts is preferred for the production of pharmaceuticals for the treatment of symptoms of osteoarthritis and rheumatoid arthritis as well as for the prevention and treatment of neoplasia, in particular adenomatous colorectal polyps in familial adenomatous polyposis, for the treatment of pain, in particular acute pain and dysmenorrhea, in particular primary dysmenorrhea.

Pharmaceutical compositions are particularly preferred, which contain the deuterated substituted pyrazolyl benzylsulfonamides according to the invention as well as their physiologically compatible salts for the treatment of symptoms of osteoarthritis and rheumatoid arthritis as well as for the prevention and treatment of neoplasia, in particular adenomatous colorectal polyps in familial adenomatous polyposis, for the treatment of pain, in particular acute pain and dysmenorrhea, in particular primary dysmenorrhea, in addition to pharmaceutically compatible adjuvants and/or additives.

The deuterated substituted pyrazolyl benzylsulfonamides according to the invention are produced analogously to known production processes for the undeuterated compounds with the use of deuterated educts with a deuteration degree of over 98%.

Thus, analogously to EP 731,795, an optionally deuterated 1-(4-methylphenyl) 1,3-butanedione is brought to reaction with an optionally deuterated 4-hydrazinobenzylsulfonamide, wherein the pyrazolyl benzylsulfonamide according to the invention which is formed contains deuterium at the positions R¹ and/or R² and/or R³ of the general formula 1, depending on the educts used.

The educts used for the production of deuterated 1-(4-methylphenyl) 1,3-butanedione and deuterated 4-hydrazinobenzylsulfonamide, such as deuterated chlorobenzene and/or deuterated 4-methylacetophenone are commercially available and also can be obtained by production processes known to the person of average skill in the art, for example, from deuterated benzene or deuterated toluene.

For the synthesis of deuterated 4-hydrazinobenzylsulfonamide, deuterated chlorobenzene is converted to deuterated 4-chlorobenzyl sulfochloride by reaction with chlorosulfonic acid and thionyl chloride analogous to EP 115,328. Without further purification, the deuterated 4-chlorobenzyl sulfochloride that is obtained can be converted to the deuterated 4-chlorobenzylsulfonamide by reaction with ammonium hydroxide solution. The deuterated 4-chlorobenzylsulfonamide thus obtained is brought to reaction with an aqueous hydrazine hydrate solution analogous to U.S. Pat. No. 3,839,325 and converted to the deuterated 4-hydrazinobenzylsulfonamide.

The production of the deuterated 1-(4-methylphenyl) 1,3-butanedione proceeds from the corresponding deuterated 4-methylacetophenones in the presence of sodium methanolate with trifluoroacetic acid ethyl ester (see e.g. EP 731,795).

Common physiologically compatible inorganic and organic acids can be used for the production of physiologically compatible salts of the deuterated substituted pyrazolyl benzylsulfonamides according to the invention. These include, for example, hydrochloric acid, hydrobromic acid, phosphoric acid, sulfuric acid, oxalic acid, maleic acid, fumaric acid, lactic acid, tartaric acid, malic acid, citric acid, salicylic acid, adipic acid and benzoic acid. Other usable acids, for example, are described in Fortschritte der Arzneimittelforschung (Advances in Pharmaceutical Research), Vol. 10, pages 224-225, Birkhäuser Publishing Co., Basel and Stuttgart, 1966, and Journal of Pharmaceutical Sciences, Vol. 66, pages 1-5 (1977).

The acid addition salts are usually obtained in a way known in and of itself by mixing the free bases or their solutions with the corresponding acids or their solutions in an organic solvent, for example, a lower alcohol such as methanol, ethanol, n-propanol or isopropanol or a lower ketone such as acetone, methyl ethyl ketone or methyl isobutyl ketone or an ether such as diethyl ether, tetrahydrofuran or dioxane. For better crystal deposition, mixtures of the named solvents can also be used. In addition, physiologically compatible aqueous solutions of acid addition salts of the compounds used according to the invention can be prepared in an aqueous acidic solution.

The acid addition salts of the compounds according to the invention can be converted into the free bases in a way known in and of itself, e.g., with alkalis or ion exchangers. From the free bases, by reaction with inorganic or organic acids, in particular those which are suitable for the formation of therapeutically usable salts, other salts can be obtained. These or even other salts of the new compound such as, e.g., the picrate, can serve also for the purification of the free base by converting the free base into a salt, separating the latter, and again releasing the base from the salt.

The subject of the present invention is also pharmaceuticals for oral, rectal, topical (percutaneous, transdermal, local), subcutaneoous, intravenous or intramuscular application, which contain, in addition to the usual vehicle and dilution agents, a compound of the general formula I or its acid addition salt as the active ingredient.

The pharmaceuticals of the invention are produced in the known way with the usual solid or liquid vehicle substances or dilution agents and the usually used pharmaceutical-technical adjuvants corresponding to the desired type of application with a suitable dosage. The preferred preparations exist in a form of administration which is suitable for oral application. Such administration forms include, for example, tablets, coated tablets, (sugar)-coated pills, capsules, pills, powders, solutions or suspensions or slow-release forms.

Topical application can be conducted, for example, in the form of salves, creams, gels, solutions, or by (adhesive) plasters.

Of course, parenteral preparations such as injection solutions are also considered. In addition, suppositories can also be named, for example, as preparations.

Corresponding tablets can be obtained, for example, by mixing the active ingredient with known adjuvants, for example, inert dilution agents such as dextrose, sugar, sorbitol, mannitol, polyvinylpyrrolidone, bursting agents such as corn starch or alginic acid, binding agents such as starch or gelatins, lubricants such as magnesium stearate or talcum and/or agents for obtaining a slow-release effect, such as carboxypolymethylene, carboxymethylcellulose, cellulose acetate-phthalate or polyvinyl acetate. The tablets can also comprise several layers.

Correspondingly, (sugar-)coated pills can be produced by coating cores, which are produced analogously to the tablets, with the agents usually employed in coating these pills, for example, polyvinylpyrrolidone or shellac, gum arabic, talcum, titanium dioxide or sugar. The envelope of the pill may also consist of several layers, whereby the above-mentioned adjuvants for tablets may be used.

Solutions or suspensions containing the active ingredient used according to the invention may additionally contain agents that improve taste such as saccharin, cyclamate or sugar, as well as, e.g., flavorings such as vanilla or orange extract. They may additionally contain suspension adjuvants such as sodium carboxymethylcellulose or preservatives such as p-hydroxybenzoate. Capsules containing active ingredients may be produced, for example, by mixing the active ingredient with an inert carrier such as milk sugar or sorbitol and encapsulating in gelatin capsules.

Suitable suppositories can be prepared, for example, by mixing with support agents provided for this purpose, such as neutral fats or polyethylene glycol or their derivatives.

The production of the pharmaceuticals according to the invention for topical application is known to the person skilled in the art. In the production of the pharmaceuticals according to the invention for transdermal application, adjuvants and enhancer substances that are known in and of themselves are employed. The production of the pharmaceutical preparations according to the invention is known in and of itself and is described in handbooks known to the person skilled in the art, for example Hager's Handbuch (Handbook) (5^{th} ed.) 2, 622-1045; List et al., Arzneiformenlehre (Study of pharmaceutical forms), Stuttgart: Scientific Publishing Co. 1985; Sucker et al., Pharmazeutische Technologie [Pharmaceutical Technology], Stuttgart: Thieme 1991; Ullmann's Enzyklopädie (Encyclopedia) (5^{th} ed.) A 19, 241-271; Voigt, Pharmazeutische Technologie (Pharmaceutical Technology), Berlin: Ullstein Mosby 1995.

The compounds substituted with deuterium targeted according to the invention have a number of advantages when compared with the known compounds of the prior art, which contain deuterium only in the natural distribution. First of all, metabolism in the organism is slowed down due to the deuteration. Because of this, it is possible to change the dosage and to create preparations that are effective over a longer period of time, which can also improve compliance in the form of slow-release preparations.

In addition, the pharmacodynamics are also changed, since the deuterated compounds according to the invention form different hydrate envelopes, so that their distribution in the organism differs from the undeuterated compounds.

It is thus possible to develop novel forms of preparation.

The following examples explain the invention:

### EXAMPLE 1

### Production of 4-chloro-2,3,5,6-tetradeuterobenzylsulfonyl chloride

11.76 g of chloropentadeuterobenzene are added by drops to a mixture of 12.23 g of chlorosulfonic acid, 15 g of thionyl chloride and 0.1 g of dimethylformamide at 80° C. while stirring within a time of 2 hours. After the addition has been completed, stirring is continued for 30 minutes while maintaining the temperature.

The reaction batch is cooled to room temperature and 21 g of crude product are obtained, which is further reacted without additional purification.

### EXAMPLE 2

### Production of 4-chloro-2,3,5,6-tetradeuterobenzylsulfonamide

21 g of the crude 4-chloro-2,3,5,6-tetradeuterobenzylsulfonyl chloride obtained in Example 1 are melted in a dropping funnel heated to 50-60° C. and added to a mixture of 40 ml of aqueous 25% ammonium hydroxide solution and 72 ml of water within 2 hours. After the addition has been completed, stirring is continued for 30 minutes while cooling to 30-35° C. The reaction batch is filtered and in this way, the deuterated 4,4′-dichlorodiphenylsulfone that is formed as a byproduct in Example 1 is separated.

The filtrate is brought to pH 5-6 by addition of hydrochloric acid, whereby the temperature of the reaction batch is maintained at 20-25° C. by means of cooling. The precipitated reaction product is separated, washed with water and dried. 17.25 g of product are obtained as a white solid.

Melting point: 143° C.

Yield: 88%, referred to 1-chloro-2,3,4,5,6-pentadeuterobenzene of Example 1

Theoretical: C, 36.83%; H, 5.15%; N, 7.16%

Experimental: C, 36.78%; H, 5.23%; N, 7.25%

¹³C-NMR (200 MHz, CDCl₃): δ 126.40 (t); 128.80 (t); 136.60 (s); 137.00 (s).

### EXAMPLE 3

### Production of 2,3,5,6-tetradeutero-4-hydrazinobenzylsulfonamide

29.35 g of 4-chloro-2,3,5,6-tetradeuterobenzylsulfonamide are added slowly while stirring to a mixture of 200 ml of dimethyl sulfate and 85 ml of an aqueous 85% hydrazine hydrate solution. The reaction batch is heated to reflux for 15 hours. After the addition of 0.2 g of activated carbon, it is stirred for another 10 minutes and then the solution which is still hot is filtered. The filtrate is immediately diluted with 550 ml of water heated to 90° C. and the solution is slowly cooled. The precipitated product is separated by filtration, washed with water and dried.

22.75 g of 2,3,5,6-tetradeutero-4-hydrazinobenzylsulfonamide are obtained as a white solid. Melting point: 156-158° C.

Yield: 79%

Theoretical: C, 37.68%; H, 6.85%; N, 21.97%

Experimental: C, 37.75%; H, 6.77%; N, 22.08%

¹³C-NMR (200 MHz, CDCl₃): δ 112.00 (t); 126.40 (t); 130.10 (s); 145.80 (s).

### EXAMPLE 4

### Production of 4,4,4-trifluoro-1-(4-trideuteromethyl-2,3,5,6-tetradeuterophenyl) 1,3-butanedione

5.65 g of 4-(trideuteromethyl)-2,3,5,6-tetradeuteroacetophenone are dissolved in 25 ml of methanol and mixed with 12.25 ml of a 25% solution of sodium methanolate in methanol under argon. The mixture is stirred for 5 minutes and then mixed with 5.6 ml of trifluoroacetic acid ethyl ester. After it has been heated to reflux for 24 hours, the reaction batch is cooled to room temperature, concentrated, and mixed with 100 ml of 10% hydrochloric acid. The solution is extracted 6×, each time with 50 ml of acetic acid ethyl ester, the organic phase is separated, dried, and the solvent is removed. 8.65 g of product are obtained as a brown oil, which is further processed without additional purification.

Yield: 91%

### EXAMPLE 5

### Production of 2,3,5,6-tetradeutero-4-[5-(4-trideuteromethyl-2,3,5,6-tetradeuterophenyl)-3-trifluoro-methyl-pyrazol-1-yl]benzylsulfonamide

4.27 g of 4,4,4-trifluoro-1-(4-trideuteromethyl-2,3,5,6-tetradeuterophenyl)-1,3-butanedione are dissolved in 75 ml of absolute ethanol and mixed with 3.63 g of 2,3,5,6-tetradeutero-4-hydrazinobenzylsulfonamide. The reaction batch is heated to reflux for 24 hours under argon and then cooled to room temperature and filtered. The solution is concentrated and the orange-colored solid that remains behind is recrystallized from a mixture of dichloromethane and hexane. 2.85 g of product are isolated as a pale yellow solid.

Melting point: 149-153° C.

Yield: 40%

Theoretical: C, 52.03%; H, 6.42%; N, 10.71%

Experimental: C, 52.38%; H, 6.57%; N, 10.66%

¹³C-NMR (200 MHz, CDCl₃): δ 20.50 (sept); 106.40 (s); 118.80 (t); 121.00 (s); 126.60-127.10 (m); 129.50 (t); 133.00 (s); 136.20 (s); 137.20 (s); 145.10 (s); 145.40 (s).

1. Deuterated substituted pyrazolyl benzylsulfonamides of the general formula I, 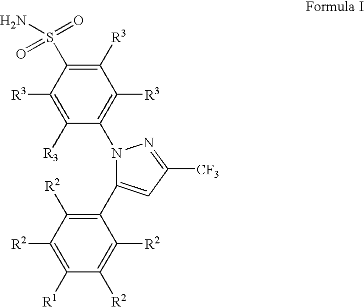
wherein
R¹ is methyl or partially or completely deuterated methyl,
R², independent of one another, indicates H or D,
R³, independent of one another, is H or D, and
wherein
at least one of the groups R¹ to R³ is D or contains D.

2. Deuterated substituted pyrazolyl benzylsulfonamides according to claim 1, wherein
R¹ is partially or completely deuterated methyl,
R², independent of one another, indicates H or D, and
R³, independent of one another, is H or D.

3. Deuterated substituted pyrazolyl benzylsulfonamides according to claim 1, wherein
R¹ is methyl or partially or completely deuterated methyl,
R² indicates deuterium and
R³, independent of one another, is H or D.

4. Deuterated substituted pyrazolyl benzylsulfonamides according to claim 1, wherein
R¹ is methyl or partially or completely deuterated methyl,
R², independent of one another, indicates H or D, and
R³ is deuterium.

5. Deuterated substituted pyrazolyl benzylsulfonamides according to claim 1, wherein
R¹ is partially or completely deuterated methyl,
R² indicates deuterium and
R³, independent of one another, is H or D.

6. Deuterated substituted pyrazolyl benzylsulfonamides according to claim 1, wherein
R¹ is methyl or partially or completely deuterated methyl, and
R² and R³ indicate deuterium.

7. Deuterated substituted pyrazolyl benzylsulfonamides according to claim 1, wherein
R¹ is partially or completely deuterated methyl,
R², independent of one another, indicates H or D, and
R³ is deuterium.

8. Deuterated substituted pyrazolyl benzylsulfonamides according to claim 1, wherein
R¹ is partially or completely deuterated methyl and
R² and R³ indicate deuterium.

9. 4-[5-(4-trideuteromethylphenyl)-3-trifluoromethylpyrazol-1-yl]benzylsulfonamide.

10. 4-[5-(2,3,5,6-tetradeutero-4-methylphenyl)-3-trifluoromethylpyrazol-1-yl]benzylsulfonamide.

11. 2,3,5,6-tetradeutero-4-[5-(4-tolyl)-3-(trifluoromethyl)pyrazol-1-yl]benzylsulfonamide.

12. 4-[5-(2,3,5,6-tetradeutero-4-trideuteromethylphenyl)-3-trifluoromethylpyrazol-1-yl]benzylsulfonamide.

13. 2,3,5,6-tetradeutero-4-[5-(2,3,5,6-tetradeutero-4-methylphenyl)-3-trifluoromethylpyrazol-1-yl]benzylsulfonamide.

14. 2,3,5,6-tetradeutero-4-[5-(4-trideuteromethylphenyl)-3-trifluoromethylpyrazol-1-yl]benzylsulfonamide.

15. 2,3,5,6-tetradeutero-4-[5-(2,3,5,6-tetradeutero-4-trideuteromethylphenyl)-3-trifluoro-methyl-pyrazol-1-yl]benzylsulfonamide.

16. Use of the deuterated substituted pyrazolyl benzylsulfonamides according to claim 1 as well as their physiologically compatible salts for the treatment of symptoms of osteoarthritis and rheumatoid arthritis as well as for the prevention and treatment of neoplasia, in particular adenomatous colorectal polyps in familial adenomatous polyposis, for the treatment of pain, in particular acute pain and dysmenorrhea, in particular primary dysmenorrhea.

17. Use of the deuterated substituted pyrazolyl benzylsulfonamides according to claim 1 as well as their physiologically compatible salts, for the production of pharmaceuticals for the treatment of symptoms of osteoarthritis and rheumatoid arthritis as well as for the prevention and treatment of neoplasia, in particular adenomatous colorectal polyps in familial adenomatous polyposis, for the treatment of pain, in particular acute pain and dysmenorrhea, in particular primary dysmenorrhea.

18. Pharmaceutical composition, which contains deuterated substituted pyrazolyl benzylsulfonamides according to claim 1 as well as their physiologically compatible salts for the treatment of symptoms of osteoarthritis and rheumatoid arthritis as well as for the prevention and treatment of neoplasia, in particular adenomatous colorectal polyps in familial adenomatous polyposis, for the treatment of pain, in particular acute pain and dysmenorrhea, in particular primary dysmenorrhea,
in addition to pharmaceutically compatible adjuvants and/or additives.
